(19) United States
(12) Patent Application Publication
(10) Pub. No.: US 2025/0383359 A1
(43) Pub. Date: Dec. 18, 2025

(54) COVID-19 Mucosal Antibody Assay

(71) Applicant: NantCell, Inc., Culver City, CA (US)

(72) Inventors: Courtney Fleenor, Culver City, CA (US); Patrick Soon-Shiong, Culver City, CA (US); Elizabeth Gabitzsch, Culver City, CA (US); Shiho Tanaka, Culver City, CA (US)

(73) Assignee: NantCell, Inc., Culver City, CA (US)

(21) Appl. No.: 18/005,332

(22) PCT Filed: Jun. 3, 2021
(86) PCT No.: PCT/IB2021/054887

§ 371 Date: Jan. 12, 2023

Related U.S. Application Data
(60) Provisional application No. 63/053,691, filed on Jul. 19, 2020.
(60) Provisional application No. 63/064,157, filed on Aug. 11, 2020.
(60) Provisional application No. 63/117,460, filed on Nov. 24, 2020.
(60) Provisional application No. 63/135,380, filed on Jan. 8, 2021.

(51) Int. Cl. G01N 33/68; C12Q 1/37; G01N 33/543; G01N 33/569

(52) U.S. Cl. CPC G01N 33/6854; C12Q 1/37; G01N 33/54346; G01N 33/56983; G01N 2333/165; G01N 2333/948; G01N 2469/20

(57) ABSTRACT

Methods and compositions are disclosed for inducing immunity against a virus such as a coronavirus in the mucosal tissue of a patient, include administering a vaccine composition to the patient by oral administration (e.g., nasal injection, nasal inhalation, oral inhalation, and/or oral ingestion). Compositions for assaying the presence of anti-viral antibodies induced by the administered vaccine or the presence of viral proteins in a saliva sample include an assay protocol for detecting neutralizing antibodies (e.g., IgA) against the virus in the saliva sample. Compositions include a kit including a stabilizing solution for the patient sample (e.g., saliva sample) and may also include conjugated aragonite particle beads for antibody or viral protein capture.

This application also claims the benefit of priority to the U.S. patent applications with the Ser. Nos. 63/053,691; 63/064,157; 63/117,460; and 63/135,380. Each of the above applications are incorporated by reference in its entirety, including the drawings and the sequence listings.

### INCORPORATION OF SEQUENCE LISTING

This application contains references to nucleic acid and polypeptide sequences which have been submitted concurrently herewith as the sequence listing text file “PAT.005246.WO001_ST25”, created on 26 May 2021. The file is 13 kilobytes in size. The aforementioned sequence listing is hereby incorporated by reference in its entirety pursuant to 37 C.F.R. § 1.52 (e).

### FIELD

The present disclosure relates to compositions and methods for assaying a virus or response to the virus in a patient sample, including detecting immunity to a viral infection in the patient sample, a vaccine composition targeting the virus, and administration of a vaccine to a patient.

### BACKGROUND

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

After several noteworthy coronavirus outbreaks in the recent years, including SARS and MERS, Corona Virus Disease 2019 (COVID-19) is yet another example of a serious infectious disease precipitated by a member of the corona virus family. While diagnostic tests have become available in a relatively short time, testing is not efficient, and numerous attempts to treat the disease have so far not had significant success. Most typically, patients with severe symptoms are treated to maintain respiration/blood oxygenation, and supportive treatment is provided to reduce or prevent multi-organ damage or even failure. Despite such interventions, the mortality rate is significant, particularly in elderly, immune compromised individuals, and individuals with heart disease, lung disease, or diabetes.

Thus, even though various methods of addressing symptoms in patients with COVID-19 are known in the art, all or almost all of them suffer from various disadvantages. Consequently, there is a need to provide improved detection and characterization of a patient's immune response to the virus and/or a vaccine composition, as well as improved compositions and methods that render a therapeutic effect, reduce or prevent viral entry into a cell, reduce direct and indirect toxicity of the virus to the patient, and/or produce an immune response that is effective to clear the virus from the patient.

### SUMMARY

The present disclosure provides methods and compositions for monitoring and assaying a viral infection, a vaccine, or the immune response of a vaccine in a patient or patient sample. The contemplated methods include assaying a patient sample for the presence of antibodies to a specific virus. The assay allows for the characterization of neutralizing and non-neutralizing antibodies, and the antibody isotype (e.g., IgA, IgG, or IgE) present in the sample. Exemplary samples from the patient include saliva (e.g., oral mucosa), a nasal mucosa swab, as well as a serum sample. Additionally, presently disclosed methods include assaying a patient's sample by competitive inhibition of specific human protein targets of the virus. For example, assays directed to severe acute respiratory syndrome (SARS)-like coronavirus (SARS-COV2) activity in a patient may include the in vitro detection of neutralizing anti-SARS-COV2 antibodies which inhibit the SARS-CoV2 spike(S) protein from binding the virus' target, the human angiotensin converting enzyme 2 (hACE2) protein.

The present disclosure also includes administering a vaccine composition to a patient by administering a vaccine composition to the patient by delivery to the nasal mucosa, oral mucosa, and/or alimentary mucosa of the patient. Preferably, the vaccine targets severe acute respiratory syndrome (SARS)-like coronavirus (SARS-COV2).

Notably, the disclosed anti-SARS-COV2 methods include obtaining a sample of saliva, nasal mucosa, and/or serum from the patient at a period of time after administering the vaccine. The sample may be first preserved in a stabilizing solution comprising glutaraldehyde, sodium benzoate, citric acid, propyl gallate, EDTA, zinc, actin, chitosan, parabens, sodium azide, or any combination thereof. More typically, the stabilizing solution comprises glutaraldehyde at 0.10 to 2.0% weight per volume (w/v), sodium benzoate at 0.10 to 1.0% w/v, and/or citric acid at 0.025 to 0.20% w/v. Additionally, the stabilizing solution further comprises aragonite particle beads having an average particle size of between 100 nm to 1 mm. The aragonite particle beads are capable of binding to immunoglobulin (Ig) proteins, anti-SARS-COV2 antibodies, or a SARS-COV2 viral protein. In exemplary embodiments, the aragonite particle beads are coupled to a recombinant ACE2 protein or a recombinant ACE2 alpha helix protein.

In some preferred embodiments, a recombinant human ACE2 (rhACE2) protein or peptide is immobilized on a detection surface. A patient sample is incubated with a SARS-COV2 spike(S) protein or peptide (e.g., a recombinant SARS-COV2 spike protein or peptide thereof), and the incubated S protein or peptide and sample are then together exposed to the detection surface with the immobilized ACE2 protein or peptide. If the patient sample does not contain any neutralizing anti-SARS-COV2 antibodies, the receptor binding domain (RBD) of the S protein is available to bind the ACE2 protein on the detection surface, and the bound S protein is detected using a labeled probe to the S protein. Accordingly, the presence of neutralizing (e.g., inhibiting) anti-SARS-CoV2 antibodies in the patient sample inhibits the S protein or peptide from binding the ACE2 on the detection surface, which thereby precludes any detection of the S-protein label. As such, if the label is detected by fluorescence, an increase of or presence of fluorescence is inversely correlated to the presence of neutralizing anti-SARS-COV2 antibodies.

For rapid SARS-COV2 antibody detection in a patient sample, the rhACE2 protein or peptide may be immobilized on the surface of an aragonite particle bead, or alternatively, on a flat surface suitable for immobilization of protein reagents (e.g., a polystyrene multi-well plate). The present disclosure includes a kit and method using a suitable detection surface (e.g., an aragonite particle bead or multi-well plate) pre-bound with the rhACE2 protein, peptide, or variants thereof.

In addition to or alternative to a wild type rhACE2 protein or peptide fragment, a mutant ACE2 protein or peptide may be immobilized on the detection surface to further characterize or confirm the binding capabilities of the anti-SARS-COV2 antibodies found in the patient's sample. In specific embodiments, the detection surface of the aragonite particle beads or a polystyrene plate are functionalized with a recombinant ACE2 protein having at least 85% sequence identity to SEQ ID NO:1, a recombinant alpha-helix ACE2 protein of SEQ ID NO: 2, or the recombinant alpha-helix ACE2 protein having at least one mutation selected from T27F, T27W, T27Y, D30E, H34E, H34F, H34K, H34M, H34W, H34Y, D38E, D38M, D38W, Q24L, D30L, H34A, and/D355L.

The contemplated subject matter also includes an aragonite composition formulated for binding an immunoglobulin (Ig) protein, an anti-SARS-COV2 antibody protein, or a SARS-COV2 viral protein. The aragonite composition includes a plurality of aragonite particle beads having an average particle size of between 100 nm to 1 mm, wherein the plurality of aragonite particle beads are functionalized with a moiety capable of binding to an immunoglobulin (Ig) protein, the anti-SARS-COV2 antibody protein and/or the SARS-COV2 viral protein.

Additional embodiments include analyzing the patient sample for at least one antibody selected from antibodies targeting the virus or a protein specific to the virus, wherein in the absence of antibodies or the presence of a viral protein, the method further comprises administering a booster of the vaccine to the patient.

Various objects, features, aspects and advantages of the present disclosure will become more apparent from the following detailed description of preferred embodiments, along with the accompanying drawing figures.

### BRIEF DESCRIPTION OF DRAWINGS

FIG. 1A is graph of exemplary antibody neutralization assays of serum samples assayed with SARS-COV2 S protein trimer at 2.971 nM and 1.4855 nM, according to embodiments of the present disclosure.

FIG. 1B is a graph of the percent inhibition corresponding to the exemplary antibody neutralization assays of FIG. 1A, according to embodiments of the present disclosure.

FIG. 2 is a graph showing the effect as measured by absorption at A450 on the effect of the 191V serum on SARS-COV2 S protein trimer binding to ACE2, according to embodiments of the present disclosure.

FIG. 3 is a graph showing percent inhibition of binding to ACE2 using plasma (114-P) and saliva (114-S) from patient 114, according to embodiments of the present disclosure.

### DETAILED DESCRIPTION

The contemplated subject matter includes compositions and methods for assaying the presence or absence of neutralizing antibodies (e.g., anti-SARS-COV2 antibodies) in a patient sample (e.g., saliva, nasal mucosa, alimentary mucosa, or serum), and/or the isotype of any antibody. The antibody status in the patient's sample may be used to assess the need for an additional vaccine dose (e.g., a booster dose/shot).

The contemplated subject matter includes methods for administering a vaccine to a patient by more than one route of administration to induce both local and systemic immune responses to the vaccine. The virus uses S protein to enter host cells by interaction of the S receptor binding domain (S RBD) with angiotensin-converting enzyme 2 (ACE2), an enzyme expressed broadly on a variety of cell types in the nose, mouth, gut and lungs as well as other organs, and importantly on the alveolar epithelial cells of the lung where infection is predominantly manifested.

In addition to the viral epitopes presented in a vaccine, the route of administration of the vaccine as well as the regimen for administering additional (i.e., booster) doses of the vaccine, can also affect whether or not the patient's immune response is robust enough to establish protection.

For an emerging virus such as the severe acute respiratory syndrome (SARS)-like coronavirus (SARS-COV2), the duration of immunity (both humoral and cell-mediated) in a patient recovered from a SARS-COV2 infection is not yet completely known, and furthermore, a vaccine protocol has not yet been tested across a varied population. Considering the current SARS-COV2 pandemic and the high rate of transmission for the SARS-COV2 virus, there is a need for a robust vaccination protocol and effective testing for the virus and/or immunity to the virus (e.g., presence of neutralizing anti-SARS-COV2 antibodies).

Vaccine Administration. The presently disclosed contemplated methods for inducing immunity in a patient include administering a vaccine by at least oral administration, and preferably by oral administration and by injection to the blood supply. Many vaccines are given via the intramuscular (IM) route to optimize immunogenicity with the direct delivery of the vaccine to the blood supply in the muscle to induce systemic immunity. The IM administration is typically preferred over subcutaneous (SC) injection which is more likely to have adverse reactions at the injection site than IM injections.

In addition to IM injection, induction of mucosal immunity has been reported to be essential to stop person-to-person transmission of pathogenic microorganisms and to limit their multiplication within the mucosal tissue. Furthermore, for protective immunity against mucosal pathogens, (e.g., SARS coronaviruses) immune activation in mucosal tissues instead of the more common approach of tolerance to maintain mucosal homeostasis allows for enhanced mucosal immune responses and better local protection. For example, nasal vaccination (delivery of a vaccine by nasal administration) induces both mucosal immunity as well as systemic immunity. See, e.g., Fujkuyama et al., 2012, Expert Rev Vaccines, 11:367-379 and Birkhoff et al., 2009, Indian J. Pharm. Sci., 71:729-731.

In order to induce both mucosal and systemic immunity in a patient, embodiments of the present disclosure include providing a vaccine to the patient by at least administration to the nasal mucosa, oral mucosa, and/or alimentary mucosa of the patient. In some embodiments, the routes of administration include administering the vaccine to the nasal mucosa, oral mucosa, and/or alimentary mucosa of the patient together with injection into the blood supply (e.g., intramuscular (IM), intravenous (IV), or subcutaneous (SC)). As used herein, oral administration of a vaccine composition includes nasal injection, nasal inhalation, ingestion by mouth, and administration (e.g., inhalation, ingestion, injection) to the alimentary mucosa. Preferably, the routes of administering the vaccine include oral administration selected from delivery to the alimentary mucosa, nasal injection, nasal inhalation, ingestion by mouth, or inhalation by mouth together with administration by intramuscular (IM) injection.

Notably, the vaccine administered for inducing immunity in the mucosal tissue of a patient is a SARS-COV2 vaccine. In exemplary embodiments, the SARS-COV2 vaccine (e.g., an adenovirus construct) includes a soluble ACE2 protein coupled to an immunoglobulin Fc portion, forming an ACE2-Fc hybrid construct that may also include a J-chain portion, as disclosed in U.S. Ser. No. 16/880,804 and U.S. 63/016,048, the entire contents of both of which are herein incorporated by reference. In other exemplary embodiments, the SARS-COV2 vaccine (e.g., an adenovirus construct) includes a mutant variant of a recombinant soluble ACE2 protein (e.g., SEQ ID NO: 2), wherein the mutant variant has at least one mutated amino acid residue (e.g., by substitution) that imparts an increased binding affinity of the ACE2 protein for the RBD protein domain of the SARS-COV2 spike protein as disclosed in U.S. 63/022,146, the entire content of which is herein incorporated by reference. In another exemplary embodiment, the SARS-COV2 vaccine (e.g., an adenovirus construct) includes a CoV2 nucleocapsid protein or a CoV2 spike protein fused to an endosomal targeting sequence (N-ETSD), as disclosed in U.S. Ser. No. 16/883,263 and U.S. 63/009,960, the entire contents of both of which are herein incorporated by reference. Additionally or alternatively, the SARS-COV2 vaccine includes modified yeast cells (e.g., Saccharomyces cerevisiae) genetically engineered to express coronaviral spike proteins on the yeast cell surface thereby creating yeast presenting cells to stimulate B cells (e.g., humoral immunity) as disclosed in U.S. 63/010,010.

In some embodiments, more than one vaccine composition as disclosed herein may be administered to a patient to induce immunity to SARS-COV2. For example, a patient may be administered genetically modified yeast cells expressing corona viral spike proteins as a single type of vaccine, or the genetically modified yeast cells may be administered together or concurrently with one or more SARS-COV2 adenovirus constructs as disclosed herein.

Monitoring presence of antibodies. The present disclosure includes monitoring or assessing a patient's immune response to either an administered vaccine (e.g., by oral administration and/or injection into the blood supply as disclosed herein) or to infection by the virus. In particular, disclosed herein are compositions and methods for assessing the continued presence of antibodies in a patient's respiratory and digestive mucosa following infection with SARS-COV2 or following inoculation against SARS-COV2 with administration of a SARS coronavirus vaccine.

For assaying a sample from a patient having received a vaccine against a pathogenic infection (e.g., targeting SARS-COV2) and/or having been infected with a virus (e.g., SARS-CoV2), the presence of antibodies against the pathogen may be carried out using any one of many diagnostic tests. In some embodiments, the diagnostic test is a cell viability assay that allows for the detection of antibodies in the presence of antigen. Exemplary diagnostic tests using a cell viability assay for anti-SARS-COV2 antibody detection are disclosed in U.S. 63/053,691, the entire contents of which are herein incorporated by reference. The cellular diagnostic assay relies on the expression of the target receptor for a given pathogen (e.g., ACE2 for SARS-COV2 infection) on the surface of an immune effector cell line (e.g., killer T cells, natural killer cells, NK-92® cells and derivatives thereof, etc.) and the expression of the pathogen ligand (e.g., Spike proteins for SARS-COV2 infection) on the surface of a surrogate cell line (e.g., HEK293 cells or SUP-B15 cells).

Additional diagnostic tests using recombinant protein variants of the ACE2 protein (the human receptor targeted by SARS-COV2 spike protein) are disclosed in U.S. Ser. No. 16/880,804, the entire contents of which are herein incorporated by reference.

In some preferred embodiments, a recombinant human ACE2 (rhACE2) protein or peptide is immobilized on a detection surface. A patient sample is incubated with a SARS-COV2 spike(S) protein or peptide (e.g., a recombinant SARS-COV2 spike protein or peptide thereof), and the incubated S protein or peptide and sample are then together exposed to the detection surface with the immobilized ACE2 protein or peptide. If the patient sample does not contain any neutralizing anti-SARS-COV2 antibodies, the receptor binding domain (RBD) of the S protein is available to bind the ACE2 protein on the detection surface, and the bound S protein is detected using a labeled probe to the S protein. Accordingly, the presence of neutralizing (e.g., inhibiting) anti-SARS-CoV2 antibodies in the patient sample inhibits the S protein or peptide from binding the ACE2 on the detection surface, which thereby precludes any detection of the S-protein label. As such, if the label is detected by spectrophotometry, an increase of or presence of signal (color, fluorescence, luminescence) is inversely correlated to the presence of neutralizing anti-SARS-COV2 antibodies.

For rapid SARS-COV2 antibody detection in a patient sample, the rhACE2 protein or peptide may be immobilized on the surface of an aragonite particle bead, or alternatively, on a flat surface suitable for immobilization of protein reagents (e.g., a polystyrene multi-well plate). The present disclosure includes a kit and method using a suitable detection surface (e.g., an aragonite particle bead or multi-well plate) pre-bound with the rhACE2 protein, peptide, or variants thereof.

In addition to or alternative to a wild type rhACE2 protein or peptide fragment, a mutant ACE2 protein or peptide may be immobilized on the detection surface to further characterize or confirm the binding capabilities of the anti-SARS-COV2 antibodies found in the patient's sample. In specific embodiments, the detection surface of the aragonite particle beads or a polystyrene plate are functionalized with a recombinant ACE2 protein having at least 85% sequence identity to SEQ ID NO:1, a recombinant alpha-helix ACE2 protein of SEQ ID NO: 2, or the recombinant alpha-helix ACE2 protein having at least one mutation selected from T27F, T27W, T27Y, D30E, H34E, H34F, H34K, H34M, H34W, H34Y, D38E, D38M, D38W, Q24L, D30L, H34A, and/D355L. In further embodiments, a soluble ACE2 protein variant having enhanced binding affinity to the RBD of the SARS-COV2 S protein may be used to determine binding affinity and/or competitive inhibition of any anti-SARS-COV2 antibodies in the patient's sample. See, e.g, Chan et al., 2020, Science, 369:1261-1265, the entire content of which is herein incorporated by reference.

Antibody testing in saliva samples. In order to more easily monitor a patient for the presence of anti-pathogen antibodies, assaying a saliva sample from the patient allows for expedited sample collection, increased patient participation, and may allow for the patient to obtain the sample themselves and either mail or transport the sample to the lab for testing. However, in order to assay saliva for the presence of neutralizing antibodies against SARS-COV2, it may be necessary to stabilize proteins in the saliva against degradation during transport and storage after sample collection prior to testing.

Upon collection of the saliva sample, the saliva is placed into a preservative solution to stabilize the components (e.g., anti-SARS COV2 antibody or viral spike protein) therein. Preservatives for biological samples are disclosed, for example, in Cunningham & al. (2018) report (“Effective Long-term Preservation of Biological Evidence,” U.S. Department of Justice grant #2010-DN-BX-K193) and U.S. Pat. No. 6,133,036 to Putcha et al. For example, a stabilizing preservative solution for a patient's saliva sample may include any one of glutaraldehyde, sodium benzoate, citric acid, propyl gallate, EDTA, zinc, actin, chitosan, parabens, sodium azide, and any combination thereof.

In specific embodiments, saliva samples may be mixed with stabilizing preservative solutions of glutaraldehyde to achieve a final glutaraldehyde concentration between 0.1% (w/v) and 2.0% (w/v), for example about 0.2% (w/v), about 0.3% (w/v), about 0.4% (w/v), about 0.5% (w/v), about 0.6% (w/v), about 0.7% (w/v), about 0.8% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), or about 1.9% (w/v).

In additional or alternative embodiments, saliva samples may be mixed with a stabilizing preservative solution of about 0.10% to about 1.00% sodium benzoate (weight/volume of sample) and/or about 0.025% to about 0.20% citric acid (weight/volume of sample). For example, the saliva sample may be mixed with 0.10%, 0.20%, 0.30%, 0.40%, 0.50%, 0.60%, 0.70%, 0.80%, 0.90%, or 1.00% w/v sodium benzoate. In additional embodiments, the saliva sample is mixed a stabilizing preservative solution of at least 0.5 mg/mL (for example, at least 0.6 mg/mL, at least 0.7 mg/mL, at least 0.8 mg/mL, at least 0.9 mg/mL, at least 1 mg/mL, at least 1.5 mg/mL, at least 2 mg/mL, at least 2.5 mg/mL, at least 3 mg/mL, at least 3.5 mg/mL, at least 4 mg/mL, at least 4.5 mg/mL, or even 5 mg/mL) of benzoic acid and/or at least 0.2 mg/mL (for example, at least 0.2 mg/mL, at least 0.25 mg/mL, at least 0.3 mg/mL, at least 0.35 mg/mL, at least 0.40 mg/mL, at least 0.50 mg/mL, at least 0.75 mg/mL, at least 1.0 mg/mL, at least 1.25 mg/mL, at least 1.5 mg/mL, at least 1.75 mg/mL, or even 2.0 mg/mL) of citric acid. As used herein, “benzoic acid” is interchangeable with benzoate salt (e.g., sodium benzoate) and “citric acid” is interchangeable with citrate salt (e.g., sodium citrate).

The saliva samples with preservatives as described above are stable for storage at temperatures between 15° C. and 40° C. for at least one hour (e.g., at least 5 hours, at least 10 hours, at least 12 hours, at least 24 hours, at least 36 hours, or at least 48 hours). Therefore, disclosed herein is a method of preserving a saliva sample for neutralizing antibody testing, the method including mixing the saliva sample with the stabilizing solution made of one or more of glutaraldehyde, sodium benzoate, citric acid, propyl gallate, EDTA, zinc, actin, chitosan, parabens, and/or sodium azide and storing between 15° C. and 25° C. for at least one hour, and up to 2, 3, 4, 5, 6, 7, 8, 9, 10, 11, 12, 13, 14, 15, 16, 17, 18, 19, 20, 21, 22, 23, 24, 36, or 48 hours. In some embodiments, the saliva sample is mixed with a glutaraldehyde concentration between 0.1% (w/v) and 2.0% (w/v), and the glutaraldehyde-saliva is stored between 15° C. and 25° C. In certain embodiments, the glutaraldehyde-saliva may further comprise citric acid and/or benzoic acid at a concentration of as disclosed herein.

Aragonite. In some embodiments, any antibody proteins or any specific antibody protein may be captured from the saliva sample with oolitic aragonite particles. For example, the saliva preserving solution of glutaraldehyde, sodium benzoate and citric acid, propyl gallate, EDTA, zinc, actin, chitosan, parabens, sodium azide, and any combination thereof as disclosed herein, may also include oolitic aragonite (calcium carbonate, CaCO₃) particles. Use of aragonite particles for binding to proteins is disclosed, for example, in U.S. Ser. No. 16/858,548 and PCT/US20/29949, the entire contents of both of which are herein incorporated by reference. Accordingly, aragonite particles may be added that have been modified to capture (e.g., bind to) any antibodies present in the saliva sample or specifically capture an antibody against a specific antigen. For example, aragonite may be functionalized with moieties capable of binding to an immunoglobulin (Ig) protein. Preferably, the Ig protein is an immunoglobulin A (IgA), immunoglobulin G (IgG), or immunoglobulin E (IgE) protein. More preferably, the aragonite is functionalized to bind to an IgA protein. Most preferably, the aragonite particles are functionalized with moieties capable of binding to specific antibodies. For example, the aragonite particles may be coupled with a moiety specific to anti-SARS-COV2 antibodies. Preferably, the aragonite particle is coupled with a recombinant ACE2 protein as disclosed, for example, in U.S. Ser. No. 16/880,804, supra. In typical embodiments, the aragonite particle is coupled with a recombinant human ACE2 protein having at least 85%, at least 90%, or at least 95% sequence identity to SEQ ID NO: 1.

In additional or alternative embodiments, the aragonite particle is functionalized to (e.g., coupled to) a recombinant soluble ACE2 protein (e.g., SEQ ID NO: 2). For more efficient capture or binding of an anti-SARS-COV2 antibody or the spike protein of SARS COV-2, the recombinant soluble ACE2 may be mutated to form ACE2 variants having higher binding affinities for SARS-CoV2 spike protein (e.g., the RBD domain of the spike protein). These ACE2 variant mutants of the recombinant soluble ACE2 protein include T27F, T27W, T27Y, D30E, H34E, H34F, H34K, H34M, H34W, H34Y, D38E, D38M, D38W, Q24L, D30L, H34A, and/or D355L.

As used herein, the term “functionalized” refers to coupling or binding of a moiety to the aragonite particle thereby imparting any function of the coupled moiety to the aragonite particle. For example, the aragonite particle may be functionalized with a protein moiety. Methods for preparing and using aragonite particle beads are disclosed in U.S. Ser. No. 16/858,548 and PCT/US20/29949. In some embodiments, the aragonite composition includes a plurality of aragonite particle beads. Preferably, the plurality of aragonite particle beads have an average particle size of between 100 nm to 1 mm,

In some embodiments a protein moiety is coupled directly to the natural, untreated surface of aragonite particles. Aragonite particles have approximately 2-3% amino acid content including aspartic acid and glutamic acid rendering the aragonite surface hydrophilic. Accordingly, in some embodiments, protein moieties may be directly coupled to the surface of the aragonite particles.

In alternative embodiments, the aragonite particle surface may be treated to modify the binding surface. For example, treatment with stearic acid (i.e., octadecanoic acid) provides for a hydrophobic surface, as disclosed in U.S. Ser. No. 16/858,548 and PCT/US20/29949. For protein loading, treatment of the aragonite with phosphoric acid forms lamellar structures. Additional conjugation techniques for coupling reactive groups to the amino acid surface of aragonite are known in the art as disclosed, for example, in Bioconjugate Techniques, Third Edition, Greg T. Hermanson, Academic Press, 2013.

Monitoring of Vaccine Protocol. Patients who do not show sufficient titers of (e.g., presence of) neutralizing antibody in their saliva may be sent oral dosages of the respective vaccine (e.g., a SARS-COV2 vaccine as disclosed herein). The patients inhale or ingest these vaccine dosages, and then two weeks later send another saliva sample—prepared and stored in the same manner as above—to the test facility to confirm that the oral vaccine dose has restored their anti-SARS-COV2 antibody (e.g., IgA) titers.

Accordingly, in additional embodiments, a kit for collecting a saliva sample from a patient includes a collection container with the saliva preservative solution as disclosed herein. For example, the kit includes a collection container with a solution of any of one or combination of glutaraldehyde, sodium benzoate and/or citric acid, propyl gallate, EDTA, zinc, actin, chitosan, parabens, and sodium azide. The kit may also include adhesive packaging and/or mailing supplies in order to secure the collection container with the saliva sample for transport or mailing. In some embodiments, the kit may also include at least one dose of the vaccine for oral administration.

Recited ranges of values herein are merely intended as a shorthand referring individually to each separate value falling within the range. Unless otherwise indicated herein, each individual value is incorporated into the specification as if it were individually recited herein.

As used in the description herein and throughout the claims that follow, “a,” “an,” and “the” include plural reference unless the context clearly dictates otherwise. Also, “in” includes “in” and “on” unless the context clearly dictates otherwise. As also used herein, and unless the context dictates otherwise, “coupled to” includes both direct coupling (in which two elements that are coupled to each other contact each other) and indirect coupling (in which at least one additional element is located between the two elements). Therefore, “coupled to” and “coupled with” are synonymous.

“Comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C, . . . and N, the text should be interpreted as requiring only one element from the group, not A plus N, or B plus N, etc.

### EXAMPLES

### Example 1. The Advantageous Features of the Compositions and Methods Described Herein are Further Illustrated (but not Limited) by the Following Examples

SUP-B15 cells are transfected with a construct comprising a CMV promoter, operatively linked to a sequence encoding the SARS CoV-2 spike protein. The spike is expressed and naturally localizes to the extracellular face of the cell membrane. The transfected SUP-B15 cells are seeded into a six well dish and allowed to attach and grow to ˜60% confluency. Three wells of this six well dish are incubated with haNK cells transfected to express a CAR with an ACE2 extracellular domain, while the other three are incubated with the same haNK cells in the presence of convalescent serum from a patient recently recovered from COVID19. Following incubation for an hour at 37° C. and 5% CO2, viability is assayed. The three wells with convalescent serum have an average cell viability of 90%, while the three well without convalescent serum show an average cell viability of 50%.

Without being bound by theory, one of the advantages of this diagnostic compositions and methods disclosed herein over existing surrogate neutralizing antibody diagnostics is that the antibodies found in the patient serum could alter the ACE-2/Spike interaction by binding elsewhere on Spike (rather than a direct Spike-RBD/ACE-2 inhibition). In the presence of haNKR cells, the antibodies that are neutralizing by inhibiting ADCC would also be recognizable by this method.

### Example 2. Samples of Saliva are Collected from One or More Patients and Stored in 1.5 mL Snap-Cap Polystyrene Tubes, Each Labeled with Marks to Indicate the Identity of the Patient from which the Sample was Collected

To each tube of saliva sample, a roughly equal volume of solution is added, the solution of 2.0% weight per volume (w/v) glutaraldehyde with 1 mg/mL sodium benzoate solution. The glutaraldehyde and sodium benzoate solution added to the saliva sample results in a solution in each tube with a final concentration of about 1.0% (w/v) glutaraldehyde/0.5 mg/mL sodium benzoate. The tubes are packaged appropriately for shipment and transported to a testing facility. The approximate duration of time between sample collection and when the sample is opened at the facility for testing is no more than 48 hours, and typically no more than about 24 hours. For example, the duration of time between sample collection and sample opening at the testing site may range from about 1 minute up to no more than 48 hours, and preferably, 1 minute up to about 36 hours. Typically, the duration of time between sample collection and sample opening at the testing site may range from about 1 minute up to about 30 hours, 1 minute up to about 30 hours, 1 minute up to about 30 hours, 1 minute up to about 30 hours, 1 minute up to about 24 hours, 1 minute up to about 20 hours, 1 minute up to about 19 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 18 hours, 1 minute up to about 30 hours (e.g., at or between 18 hours to 30 hours). During the approximate 24 hours, the sample never reaches a temperature colder than about 15° C. (e.g., at or between 18 hours to 30 hours), and never hotter than about 40° C., averaging about 25° C. across the about 24 hour period (e.g., at or between 18 hours to 30 hours). As such, the sample may be at a temperature of about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

At the testing facility, technicians open each tube and extract the contents with a sterile pipette. The solution is dialyzed to remove the glutaraldehyde and benzoate. The dialyzed sample is then mixed with a peptide mimic to the SARS-COV2 spike protein receptor-binding domain (RBD) and allowed to incubate for 30 minutes at room temperature.

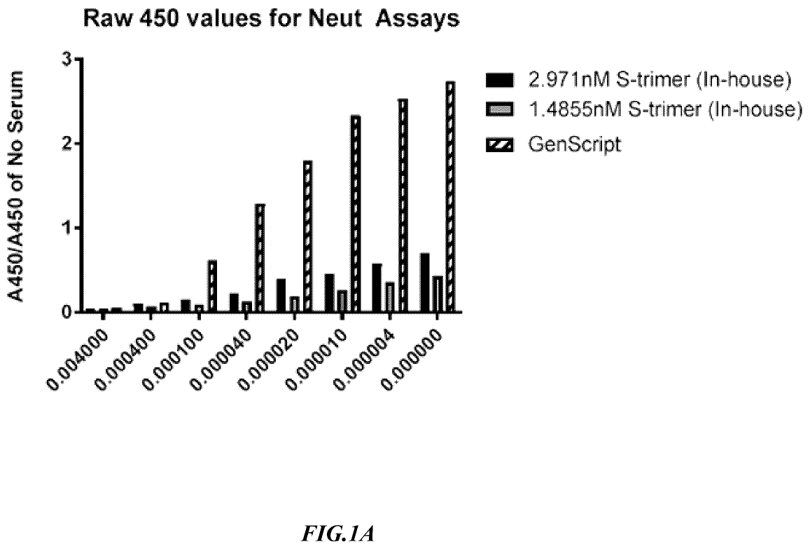

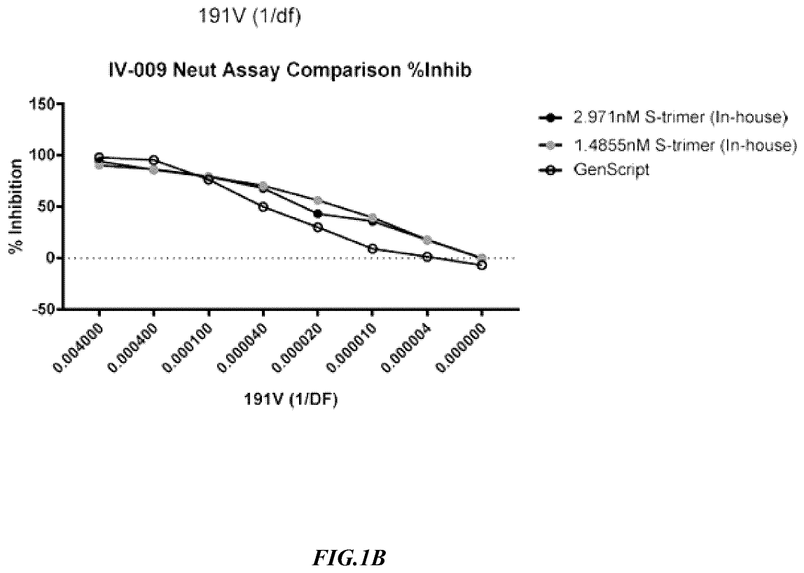

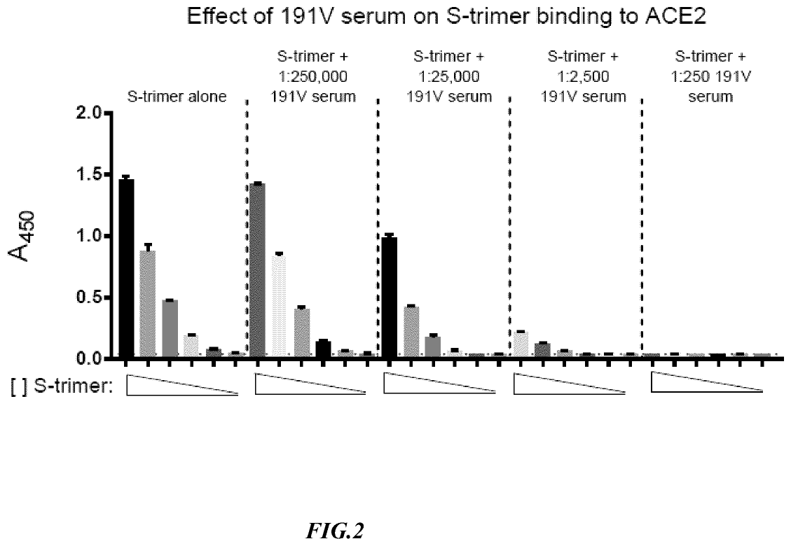

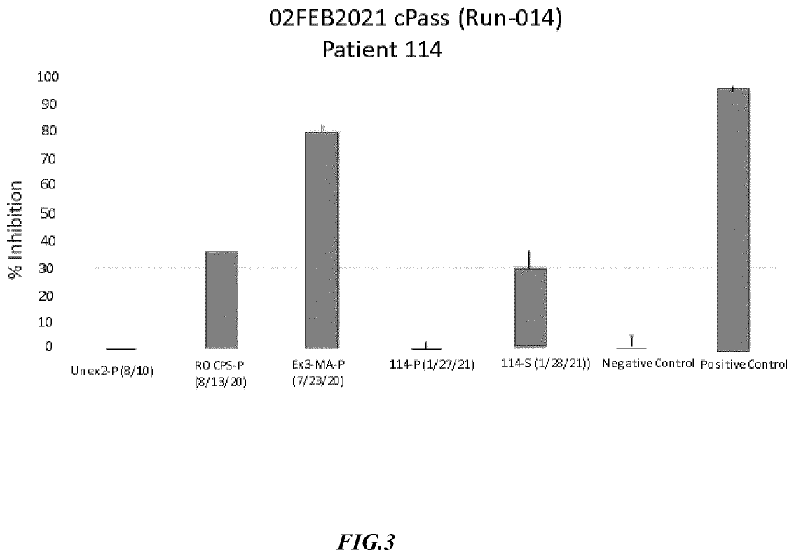

This peptide is conjugated to a horseradish peroxidase (HRP). The RBD peptide/sample (antibody) mixture is then added to a well of a multi-well polystyrene plate, the inside surfaces of which have been coated with recombinant human ACE2 (rhACE2) and allowed to incubate for 30 minutes at room temperature. At the end of the incubation, the wells are washed three times with room temperature PBS and 3,3′,5,5′-tetramethylbenzidine (TMB) is added to each well. The wells that received samples without neutralizing antibody turn dark blue, while the wells that received samples with neutralizing antibody remain clear. The darkness of the TMB can be read by spectrophotometry at OD450. Accordingly, with reference to FIGS. 1A, 1B, 2, and 3, the raw spectrophotometry data corresponds to the OD450 (or A450), in which the presence of neutralizing antibody correlates to inhibition of binding to the coated rhACE2 which results in a decrease in the OD450.

### Example 3. Exemplary Method for Assaying for Neutralizing Anti-SARS-COV2 S Antibodies In a Patient Sample

A microplate (e.g., Nunc MaxiSorp ELISA plate with 100 μl/well) is coated with antigen. Dilute human ACE2-hFC protein to 0.5 ug/mL (ImmunityBio, Inc. at 2.5 mg/mL) (˜4.5455 nM, DF=1:5,200) in coating Buffer (BioLegend Catalog No. 421701, diluted in water for injection (WFI)). Coat each well of the microplate with diluted ACE2-hFC protein. Cover the microplate with an adhesive plastic and incubate at 4° C. overnight.

The coating solution is removed from each well and washed three times (3×) with 200-250 μl PBS/well. The solutions or washes are removed by flicking the plate over a sink. The remaining drops are removed by patting the plate on a paper towel.

The coated wells are blocked by adding 200 μl/well blocking buffer (2% non-fat dry milk in DPBS).

The blocked wells are then covered with an adhesive plastic and incubate for 1 to 2 hours at 37° C. Blocking buffer is removed by flicking plate over sink. The wells are washed 3× with 200-250 uL 0.05% Tween20/PBS.

The patient sample (e.g., saliva or serum) is incubated with the SARS-COV2 S protein, peptide, or variant thereof. The SARS-COV2 S protein (e.g. SARS-COV2 S (R683A R685) trimer protein, a SARS-COV2 S (N501Y) protein, or a SARS-COV2 S (L452R) protein).

Prepare ‘2×’ sample (e.g., saliva or serum) dilutions in 2% NF Milk/PBS (60 uL volume). DF range (mouse serum)=250-250,000. For example, dilute SARS-COV-2 S Protein (R683A, R685A), active trimer, His-tagged, “S-Trimer” (Acro Biosystems, SPN-C52H9; stock @ 205 ug/mL (1.4855 uM)) in blocking buffer as indicated below in Table 1.

TABLE 1

| | Final | Final [ ] |
|---|---|---|
| Master mix S-trimer (205 μg/mL stock = 1.4855 μM) | DF | (nM) |
| 2 mL Blocking Buffer + 8 μL S-trimer | 250 | 5.94 |
| 2 mL Blocking Buffer + 4 μL S-trimer | 500 | 2.97 |
| 2 mL Blocking Buffer + 2 μL S-trimer | 1,000 | 1.49 |

Add 60 uL diluted sample 60 uL appropriate S-trimer dilution per well of 1.2 mL 96 well plate according to layout below. Mix by pipetting. Cover the plate with an adhesive plastic and incubate for 30 minutes at 37° C.

After blocking and subsequent washing of hACE2-hFC coated-wells, the sample and S trimer mixtures are incubated with the antigen (ACE2) coated-plate. For example, 100 μL/well of sample and S-trimer mixture are added and incubated for 30 minutes up to 2 hours at 37° C. After this incubation, the wells are washed up to four times (4×) with 200-250 uL 0.05% Tween20/PBS. The wells are then incubated with a detection reagent to determine the amount of S trimer bound to the wells. In this example, the S trimer is His-tagged and may be detected using an anti-HIS HRP conjugated antibody (BioLegend #_652504, @ 0.5 mg/mL; recommended use at 0.01-1 ug/mL). For detection with TMB (3,3′,5,5′-tetramethylbenzidine): add 100 μl of diluted conjugated secondary antibody solution per well. Cover the plate with an adhesive plastic cover and incubate for 1 hr at 37° C. Wash the plate 4× with 200-250 μL/well 0.05% Tween20/PBS. Mix TMB substrates (Pierce TMB substrate Kit Cat #34021) 1:1. Add 100 μL of TMB solution to each well. Incubate for up to 30 minutes at room temperature in dark. Stop reaction by adding add equal volume (100 uL) of stopping solution (Thermo #N600). Using ELISA plate reader, the optical density is read at 450 nm within 30 minutes of stop.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The present disclosure, therefore, is not to be restricted except in the scope of the appended claims. Moreover, in interpreting both the specification and the claims, all terms should be interpreted in the broadest manner consistent with the context.

1. A method of detecting presence or absence of antibodies that target the severe acute respiratory syndrome (SARS)-like coronavirus (SARS-COV2) virus in a patient sample, the method comprising:
incubating a solution of the patient sample with a recombinant SARS-COV2 S protein, peptide, or variant thereof to form a SARS-COV2 S treated sample solution;
immobilizing a recombinant human angiotensin-converting enzyme 2 (rhACE2) protein, peptide, or variant thereof on a detection surface to form an ACE2-bound detection surface;
incubating the SARS-COV2 S treated sample solution with the ACE2-bound detection surface to form a sample treated ACE2-bound detection surface in solution;
removing the solution from the sample treated ACE2-bound detection surface to remove any SARS-COV2 S protein, peptide, or variant thereof not bound to the ACE2-bound detection surface;
adding a labeled probe that binds to the SARS-COV2 S protein, peptide, or variant thereof when bound to the immobilized rhACE2 protein, peptide, or variant thereof on the detection surface; and
identifying or quantifying the labeled probe on the sample treated ACE2-bound detection surface,
wherein detecting the labeled probe on the sample treated ACE2-bound detection surface indicates an absence or decrease in the patient sample of anti-SARS-COV2 S antibodies.

2. The method of claim 1, further comprising assaying the solution removed from the sample treated ACE2-bound detection surface for presence or absence of antibody-bound SARS-COV2 S protein, peptide, or variant thereof.

3. The method of claim 1, wherein the recombinant SARS-COV2 S protein, peptide, or variant thereof is selected from a SARS-COV2 S (R683A R685) trimer protein, a SARS-CoV2 S (N501Y) protein, or a SARS-COV2 S (L452R) protein.

4. The method of claim 1, wherein the SARS-COV2 S protein comprises a detection label.

5. The method of claim 4, wherein the detection label is a histidine tag or an N-hydroxysuccinimide ester (NHS-ester) tag

6. The method of claim 1, wherein the patient sample is saliva, nasal mucosa, or serum.

7-17. (canceled)

18. An aragonite composition formulated for binding an immunoglobulin (Ig) protein or a SARS-COV2 viral protein, the aragonite composition comprising:
a plurality of aragonite particle beads having an average particle size of between 100 nm to 1 mm,
wherein the plurality of aragonite particle beads are functionalized with a moiety selected from an Ig binding protein or a SARS-COV2 virus binding protein.

19. The composition of claim 18, wherein the SARS-COV2 viral binding protein comprises a recombinant ACE2 protein.

20. The composition of claim 19, wherein the recombinant ACE2 protein has at least 85% sequence identity to SEQ ID NO:1.

21. The composition of claim 19, wherein the recombinant ACE2 protein comprises the primary sequence of SEQ ID NO:2, and optionally wherein the recombinant ACE2 protein comprises at least one mutation selected from T27F, T27W, T27Y, D30E, H34E, H34F, H34K, H34M, H34W, H34Y, D38E, D38M, D38W, Q24L, D30L, H34A, and D355L.

22. (canceled)

23. The composition of claim 18, wherein the Ig is an anti-SARS-COV2 antibody.

24-31. (canceled)


SEQUENCE LISTING (2 sequences)

SEQ ID NO: 1 - protein, 805 aa, Homo sapiens [sequence omitted]

SEQ ID NO: 2 - protein, 594 aa, Artificial Sequence
  Soluble ACE2 mutant constructs
  MISC_FEATURE (4)..(4): Can be Gln or Leu
  MISC_FEATURE (7)..(7): Can be Thr, Phe, Trp, or Tyr
  MISC_FEATURE (10)..(10): Can be Asp, Glu, Leu
  MISC_FEATURE (14)..(14): Can be His, Glu, Phe, Lys, Met, Trp, Tyr, or Ala
  MISC_FEATURE (18)..(18): Can be Asp, Glu, Met, or Phe
  MISC_FEATURE (335)..(335): Can be Asp or Leu
IEEXAKXFLXKFNXEAEXLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGXFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA